(19) United States
(12) Patent Application Publication
(10) Pub. No.: US 2025/0345569 A1
(43) Pub. Date: Nov. 13, 2025

(54) GUIDEWIRE WITH AN IMPROVED FEEL

(71) Applicant: BOSTON SCIENTIFIC SCIMED, INC., Maple Grove, MN (US)

(72) Inventors: KEVIN WALSH, Wellesly, MA (US); JOHN THOMAS FAVREAU, Charlton, MA (US); BRIAN GAFFNEY, Rutland, MA (US)

(73) Assignee: BOSTON SCIENTIFIC SCIMED, INC., Maple Grove, MN (US)

(21) Appl. No.: 19/205,274

(22) Filed: May 12, 2025

Related U.S. Application Data
(60) Provisional application No. 63/646,192, filed on May 13, 2024.

(51) Int. Cl. A61M 25/09

(52) U.S. Cl. CPC A61M 25/09; A61M 2025/09083; A61M 2025/09108; A61M 2025/09116; A61M 2025/09141; A61M 2207/00

(57) ABSTRACT

A medical device, such as a guidewire with tactile feel, is disclosed. An example medical device may include an elongate shaft having a proximal end region and a distal end region. The proximal end region of the elongate shaft may include a grip section for grasping the medical device. The grip section may have a substantially circular cross-sectional shape, and may include a flat surface, a circular segment cutout and/or a projection. In some instances, the grip section may be a twisted or helical arrangement having a length extending along at least a portion of the proximal end region.

### CROSS-REFERENCE TO RELATED APPLICATIONS

The application claims the benefit of U.S. Provisional Patent Application Ser. No. 63/646,192, filed on May 13, 2024, the disclosure of which is incorporated herein by reference.

### TECHNICAL FIELD

The present disclosure pertains to medical devices, and methods for manufacturing medical devices. More particularly, the present disclosure pertains to guidewires with an improved feel.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A medical device with tactile feel is disclosed. The medical device comprises: an elongate shaft having a proximal end region and a distal end region; wherein the proximal end region of the elongate shaft has a substantially circular cross-sectional shape; and wherein the proximal end region includes a circular segment cutout formed therein, the circular segment cutout having a length extending along at least a portion of the proximal end region.

Alternatively or additionally to any of the embodiments above, the proximal end region and the distal end region are formed from a single monolith of material.

Alternatively or additionally to any of the embodiments above, the proximal end region includes a nickel-titanium alloy.

Alternatively or additionally to any of the embodiments above, the proximal end region includes stainless steel.

Alternatively or additionally to any of the embodiments above, a polymer tip member is disposed along the distal end region.

Alternatively or additionally to any of the embodiments above, a spring tip member is disposed along the distal end region.

Alternatively or additionally to any of the embodiments above, the circular segment cutout extends helically about the proximal end region.

Alternatively or additionally to any of the embodiments above, the circular segment cutout extends axially along the proximal end region.

Alternatively or additionally to any of the embodiments above, the proximal end region includes an outer coating.

A guidewire is disclosed. The guidewire comprises: an elongate core wire having a proximal end region and a distal end region; wherein the proximal end region includes a circular segment cutout formed therein, the circular segment cutout extending along at least a length of the proximal end region; and a tip member coupled to the distal end region.

Alternatively or additionally to any of the embodiments above, the elongate core wire is formed from a single monolith of material.

Alternatively or additionally to any of the embodiments above, the elongate core wire includes a nickel-titanium alloy.

Alternatively or additionally to any of the embodiments above, the elongate core wire includes stainless steel.

Alternatively or additionally to any of the embodiments above, the tip member includes a polymer tip member disposed along the distal end region.

Alternatively or additionally to any of the embodiments above, the tip member includes a spring tip member disposed along the distal end region.

Alternatively or additionally to any of the embodiments above, the circular segment cutout extends helically about the proximal end region.

Alternatively or additionally to any of the embodiments above, the circular segment cutout extends axially along the proximal end region.

A method for manufacturing a guidewire is disclosed. The method comprises: forming a circular segment cutout in a proximal end region of an elongate core wire; wherein forming the circular segment cutout includes extending the circular segment cutout along at least a length of the proximal end region of the elongate core wire; and coupling a tip member to a distal end region of the elongate core wire.

Alternatively or additionally to any of the embodiments above, forming the circular segment cutout includes extending the circular segment cutout helically about the proximal end region.

Alternatively or additionally to any of the embodiments above, forming the circular segment cutout includes extending the circular segment cutout axially along the proximal end region.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional side view of an example medical device.

FIG. 2 is a partial cross-sectional side view of an example medical device.

FIG. 3 is a perspective view of a portion of an example medical device.

FIG. 3A is a perspective view of a portion of an example medical device.

FIG. 4 is an end view of a portion of an example medical device.

FIG. 5 is a perspective view of a portion of an example medical device.

FIG. 5A is a perspective view of a portion of an example medical device.

FIG. 6 is an end view of a portion of an example medical device.

FIG. 7 is a perspective view of a portion of an example medical device.

FIG. 8 is a perspective view of a portion of an example medical device.

FIG. 9 is an end view of a portion of an example medical device.

FIG. 10 is an end view of a portion of an example medical device.

FIG. 11 is an end view of a portion of an example medical device.

FIG. 12 is an end view of a portion of an example medical device.

FIG. 13 is a perspective view of a portion of an example medical device.

FIG. 14 is a perspective view of a portion of an example medical device.

FIG. 15 is an end view of a portion of an example medical device.

FIG. 16 is a perspective view of a portion of an example medical device.

FIG. 17 is a perspective view of a portion of an example medical device.

FIG. 18 is an end view of a portion of an example medical device.

FIG. 19 is a perspective view of a portion of an example medical device.

FIG. 20 is a perspective view of a portion of an example medical device.

FIG. 21 is an end view of a portion of an example medical device.

FIG. 22 is a perspective view of a portion of an example medical device.

FIG. 23 is a perspective view of a portion of an example medical device.

FIG. 24 is an end view of a portion of an example medical device.

FIG. 25 is a perspective view of a portion of an example medical device.

FIG. 26 is a perspective view of a portion of an example medical device.

FIG. 27 is an end view of a portion of an example medical device.

FIG. 28 is a perspective view of a portion of an example medical device.

FIG. 29 is a perspective view of a portion of an example medical device.

FIG. 30 is an end view of a portion of an example medical device.

FIG. 31 is a perspective view of a portion of an example medical device.

FIG. 32 is a perspective view of a portion of an example medical device.

FIG. 33 is an end view of a portion of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Less invasive cardiovascular interventions are used to diagnose and treat a number of different conditions. Many of these interventions utilize a guidewire, which can be advanced through the vasculature toward a target site. When advancing the guidewire, a clinician may grasp the proximal end region of the guidewire in order to apply force, rotate, and/or otherwise manipulate the guidewire. Disclosed herein are medical devices such as guidewires that have an improved feel. This may include a proximal end region with a desirable shape, texture, and/or structure that improves the feel, grip, tactile response, and/or the like, which may make it easier for a clinician to apply force, rotate, and/or otherwise manipulate the guidewire.

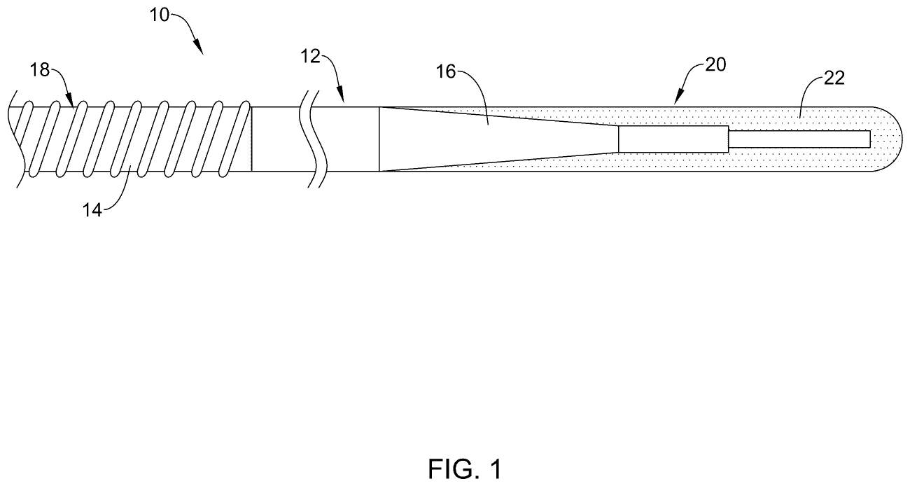

FIG. 1 illustrates an example medical device 10. In this example, the medical device 10 may include a guidewire. However, this is not intended to be limiting as other medical devices are contemplated. The guidewire 10 may an elongate shaft or core wire 12. The structure of the core wire 12 can vary. The core wire 12 may include a proximal end region 14 and a distal end region 16. In some instances, the proximal end region 14 and the distal end region 16 are formed from a single monolith of material. The material may include a nickel-titanium alloy (e.g., nitinol), stainless steel, other suitable materials including those disclosed herein, and/or the like. Alternatively, the proximal end region 14 and the distal end region 16 may be formed as separate pieces that are joined together using a suitable bonding methodology. The separate pieces may be made from the same or different materials.

A grip member or section 18 may be disposed along the core wire 12, for example along the proximal end region 14. In this example, the grip section 18 may be characterized by a projection disposed along the proximal end region 14 of the core wire 12. The projection may be arranged in a helical fashion about the core wire 12. In some instances, the grip section 18 may be formed by disposing a coil about the proximal end region 14. Alternatively, the projection forming the grip section 18 may be formed by cutting, etching, mechanically working, and/or the like. In some of these and in other instances, the grip section 18 may include a coating with a tacky or textured consistency or feel.

The distal end region 16 of the core wire 12 may include one or more tapered regions where the outer diameter thereof decreases in the distal direction. A tip member 20 may be coupled to the distal end region 16. The tip member 20 may include a polymer tip 22. In at least some instances, the polymer tip 22 may be disposed along at least some of the one or more tapers of the distal end region 16. A number of different arrangements are contemplated.

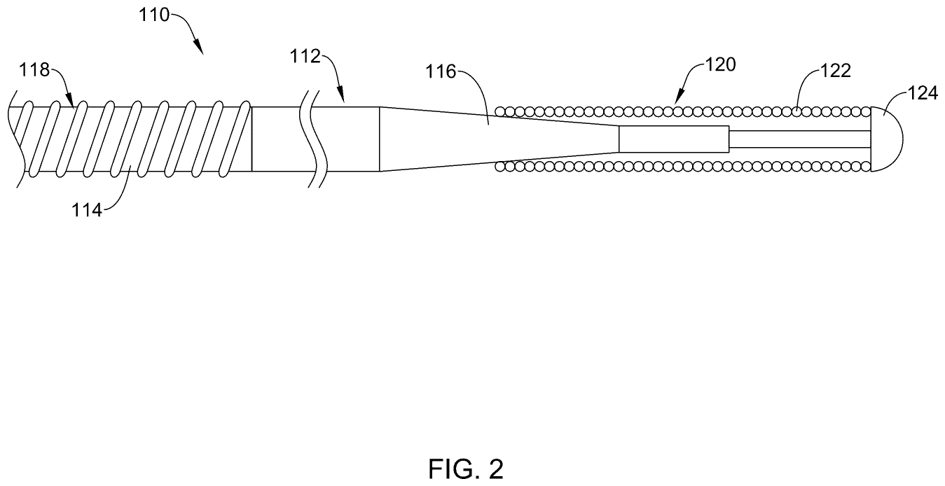

FIG. 2 illustrates another example medical device 110 that may be similar in form and function to other medical devices disclosed herein. In this example, the medical device 110 may include a guidewire. The guidewire 110 may an elongate shaft or core wire 112. The core wire 112 may include a proximal end region 114 and a distal end region 116. A grip member or section 118 may be disposed along the core wire 112, for example along the proximal end region 114. A tip member 120 may be coupled to the distal end region 116. The tip member 120 may take the form of a spring tip and may include a coil or spring 122 and a distal tip 124.

As indicated above, it may be desirable for the guidewire 10, 110 (e.g., the proximal end region 14, 114) to have a shape, texture, and/or structure that improves the feel, grip, tactile response, and/or the like. The figures and description below show/describe some of the variations contemplated for including a shape, texture, and/or structure that improves the feel, grip, tactile response, and/or the like. In general, the description is aimed at associating the features with the proximal end region of a core wire of a guidewire. However, other arrangements/devices are contemplated. For example, the new/added features may be along the entire proximal end region, a portion of the proximal end region, other locations along the device including distal portions, etc. The features may be incorporated onto a solid structure or wire, or the features may be incorporated onto a tubular structure. The features may be incorporated by removing material from the core wire, adding material, or both. The features may be added to a guidewire, a catheter, and/or the like.

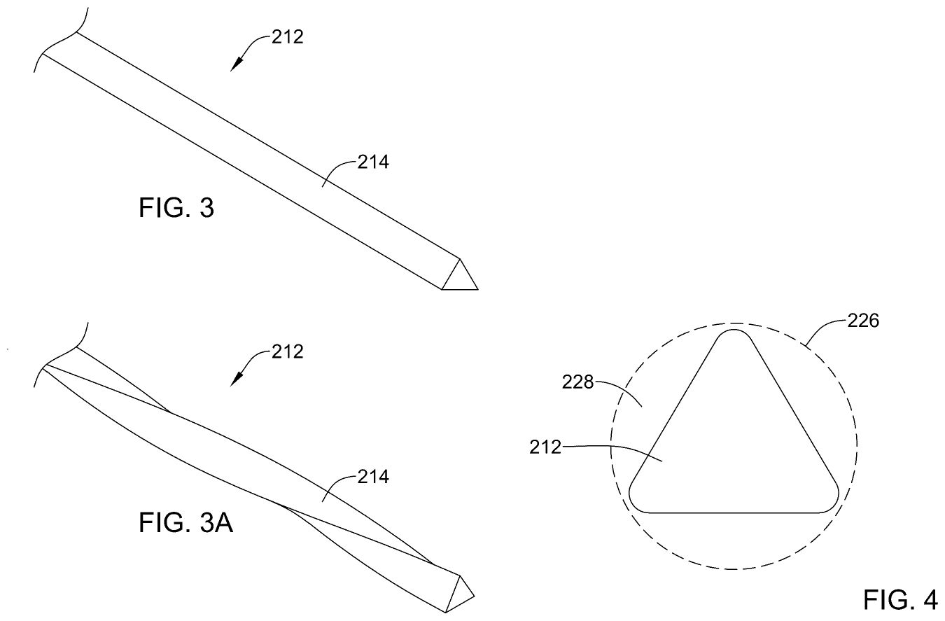

FIGS. 3-4 schematically illustrate a portion of a core wire 212 that may be used, for example, in a suitable medical device such as those disclosed herein. In at least some instances, FIGS. 3-4 may illustrate a proximal end region 214 of the core wire 212. However, this is not intended to be limiting as the portion of the core wire 212 may be essentially any suitable length or segment of the core wire 212. As can be seen in FIG. 3, the proximal end region 214 of the core wire 212 may have a geometric shape. In this example, the proximal end region 214 of the core wire 212 has a triangular cross-sectional shape (e.g., the proximal end region 214 of the core wire 212 may have a shape resembling a triangular prism). In some instances, the proximal end region 214 may have a straight configuration as shown in FIG. 3. Alternatively, the proximal end region 214 may have a twisted or helical arrangement as shown in FIG. 3A.

In at least some instances, the proximal end region 214 of the core wire 212 may be formed from a shaft or wire that has a substantially circular cross-sectional shape. For example, in FIG. 4, reference number 226 corresponds to the shape or perimeter of the core wire 212 prior to forming/cutting the geometric shape into the core wire 212. The portion of the core wire 212 that is removed to form/cut the geometric shape into the core wire 212 is labeled with reference number 228. Material may be removed from the core wire 212 using a suitable process such as laser cutting, mechanically (e.g., saw) cutting, etching, etc. After removing the material 228, the proximal end region 214 may have the desired shape. This may provide a desirable level of feel, grip, tactile response, and/or the like. In other instances, the shape of the proximal end region 214 may be achieved by molding, extruding, casting, mechanically working, or otherwise forming the proximal end region 214 into the desired shape. In some instances, the proximal end region 214 may include an outer coating with a tacky or textured consistency or feel.

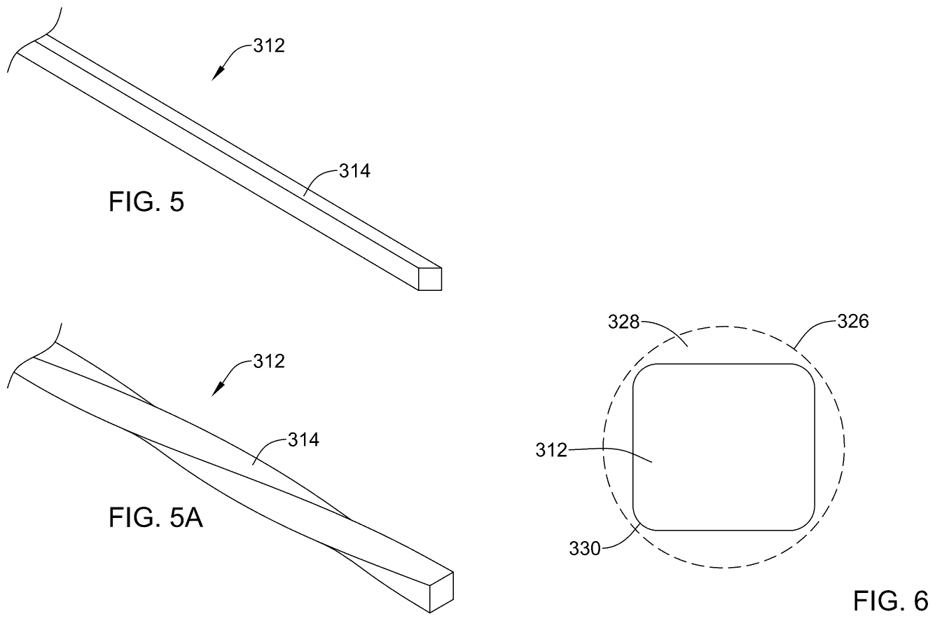

FIGS. 5-6 schematically illustrate a portion of a core wire 312 that may be used, for example, in a suitable medical device such as those disclosed herein. In at least some instances, FIGS. 5-6 may illustrate a proximal end region 314 of the core wire 312. However, this is not intended to be limiting as the portion of the core wire 312 may be essentially any suitable length or segment of the core wire 312. As can be seen in FIG. 5, the proximal end region 314 of the core wire 312 may have a geometric shape. In this example, the proximal end region 314 of the core wire 312 has a square or rectangular cross-sectional shape (e.g., the proximal end region 314 of the core wire 312 may have a shape resembling a square or rectangular prism). In at least some instances, the corners 330 of the core wire 312 may be rounded. In some instances, the proximal end region 314 may have a straight configuration as shown in FIG. 5. Alternatively, the proximal end region 314 may have a twisted or helical arrangement as shown in FIG. 5A.

In at least some instances, the proximal end region 314 of the core wire 312 may be formed from a shaft or wire that has a substantially circular cross-sectional shape. For example, in FIG. 6, reference number 326 corresponds to the shape or perimeter of the core wire 312 prior to forming/cutting the geometric shape into the core wire 312. The portion of the core wire 312 that is removed to form/cut the geometric shape into the core wire 312 is labeled with reference number 328. In other instances, the shape of the proximal end region 314 may be achieved by molding, extruding, casting, mechanically working, or otherwise forming the proximal end region 314 into the desired shape. In some instances, the proximal end region 314 may include an outer coating with a tacky or textured consistency or feel.

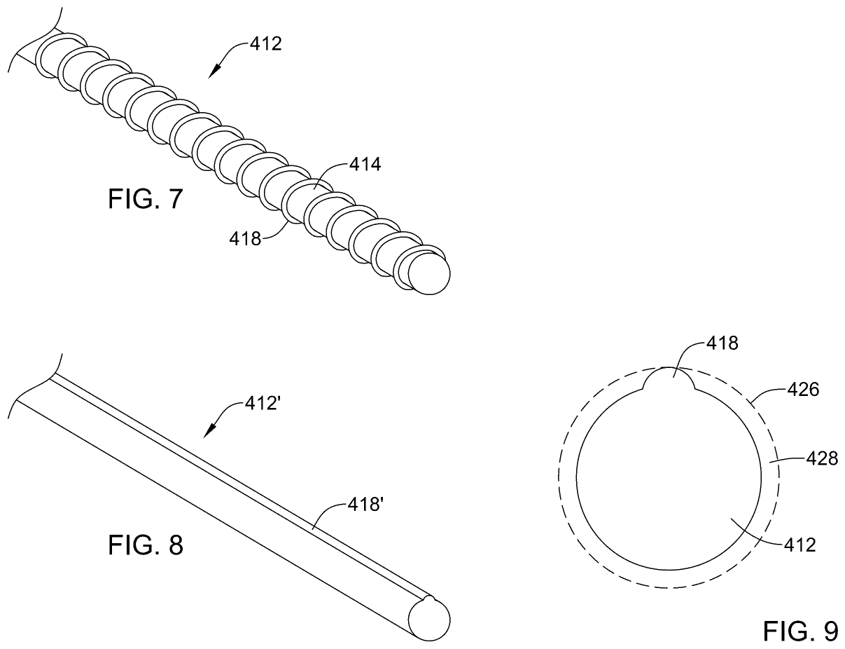

FIGS. 7-9 schematically illustrate a portion of a core wire 412 that may be used, for example, in a suitable medical device such as those disclosed herein. In at least some instances, FIGS. 7-9 may illustrate a proximal end region 414 of the core wire 412. However, this is not intended to be limiting as the portion of the core wire 412 may be essentially any suitable length or segment of the core wire 412. As can be seen in FIG. 7, the proximal end region 414 of the core wire 412 may include a projection 418. The projection 418 may extend along a length of the core wire 412. For example, the projection 418 may extend helically about the core wire 412. Alternatively, the projection 418′ may extend axially along the core wire 412′ as shown in FIG. 8.

In at least some instances, the proximal end region 414 of the core wire 412 may be formed from a shaft or wire that has a substantially circular cross-sectional shape. For example, in FIG. 9, reference number 426 corresponds to the shape or perimeter of the core wire 412 prior to forming/cutting the projection 418 in the core wire 412. The portion of the core wire 412 that is removed to form/cut the projection 418 in the core wire 412 is labeled with reference number 428. In other instances, the shape of the proximal end region 414 may be achieved by molding, extruding, casting, mechanically working, or otherwise forming the proximal end region 414 into the desired shape. In some instances, the proximal end region 414 may include an outer coating with a tacky or textured consistency or feel.

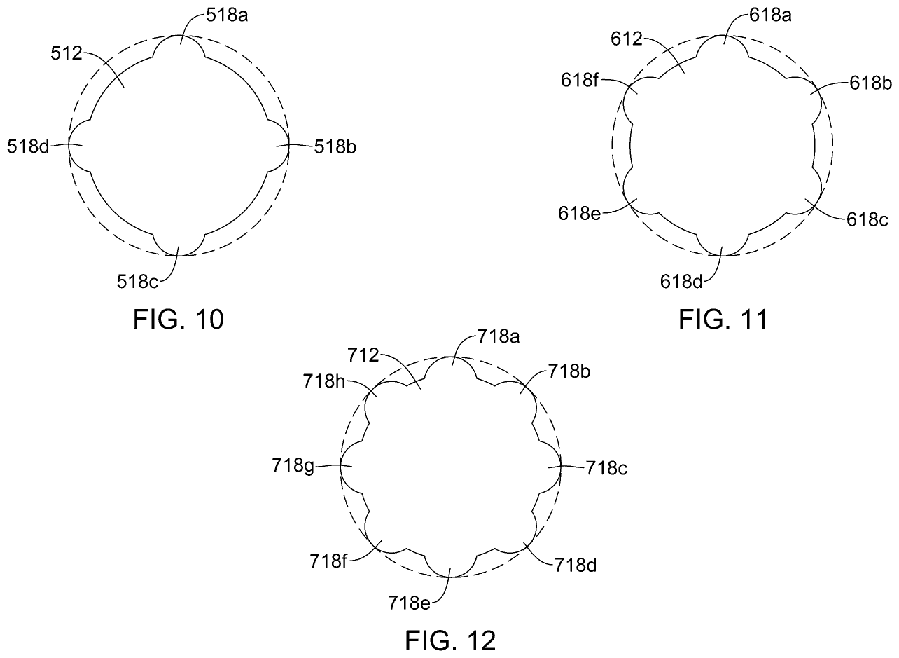

It can be appreciated that different number of projections may be utilized for various core wires. For example, FIG. 10 illustrates a portion of a core wire 512 having four projections 518a, 518b, 518c, 518d. FIG. 11 illustrates a portion of a core wire 612 having six projections 618a, 618b, 618c, 618d, 618e, 618f. FIG. 12 illustrates a portion of a core wire 712 having eight projections 718a, 718b, 718c, 718d, 718e, 718f, 718g, 718h.

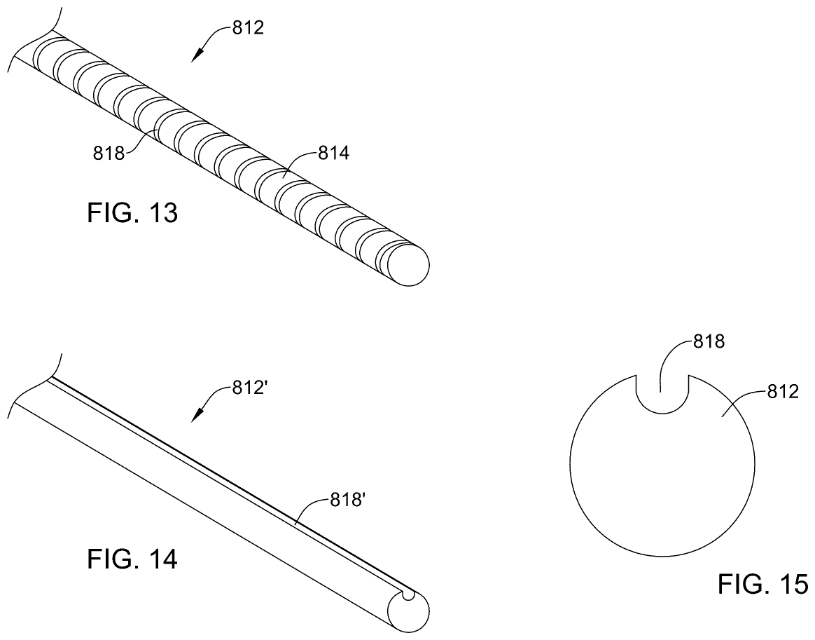

FIGS. 13-15 schematically illustrate a portion of a core wire 812 that may be used, for example, in a suitable medical device such as those disclosed herein. In at least some instances, FIGS. 13-15 may illustrate a proximal end region 814 of the core wire 812. However, this is not intended to be limiting as the portion of the core wire 812 may be essentially any suitable length or segment of the core wire 812. As can be seen in FIG. 13, the proximal end region 814 of the core wire 812 may include a groove 818. The groove 818 may extend along a length of the core wire 812. For example, the groove 818 may extend helically about the core wire 812. Alternatively, the groove 818′ may extend axially along the core wire 812′ as shown in FIG. 14.

In at least some instances, the proximal end region 814 of the core wire 812 may be formed from a shaft or wire that has a substantially circular cross-sectional shape. The groove 818 may be formed by cutting out a portion of the core wire 812 as can be seen in FIG. 15. In other instances, the shape of the proximal end region 814 may be achieved by molding, extruding, casting, mechanically working, or otherwise forming the proximal end region 814 into the desired shape. In some instances, the proximal end region 814 may include an outer coating with a tacky or textured consistency or feel.

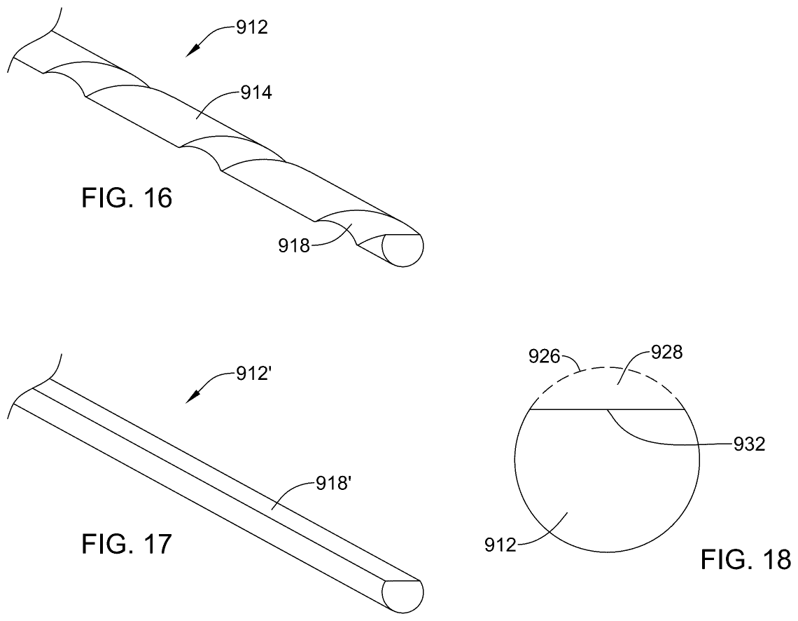

FIGS. 16-18 schematically illustrate a portion of a core wire 912 that may be used, for example, in a suitable medical device such as those disclosed herein. In at least some instances, FIGS. 16-18 may illustrate a proximal end region 914 of the core wire 912. However, this is not intended to be limiting as the portion of the core wire 912 may be essentially any suitable length or segment of the core wire 912. As can be seen in FIG. 16 the proximal end region 914 of the core wire 912 may include a circular segment cutout 918. The circular segment cutout 918 may extend along a length of the core wire 912. For example, the circular segment cutout 918 may extend helically about the core wire 912. Alternatively, the circular segment cutout 918′ may extend axially along the core wire 912′ as shown in FIG. 17.

In at least some instances, the proximal end region 914 of the core wire 912 may be formed from a shaft or wire that has a substantially circular cross-sectional shape. For example, in FIG. 18, reference number 926 corresponds to the shape or perimeter of the core wire 912 prior to forming/cutting the circular segment cutout 918 into the core wire 912. The circular segment cutout 918 may have a flattened outward-facing surface, denoted by the chord 932. The portion of the core wire 912 that is removed to form/cut the circular segment cutout 918 in the core wire 912 is labeled with reference number 928. In other instances, the shape of the proximal end region 914 may be achieved by molding, extruding, casting, mechanically working, or otherwise forming the proximal end region 914 into the desired shape. In some instances, the proximal end region 914 may include an outer coating with a tacky or textured consistency or feel.

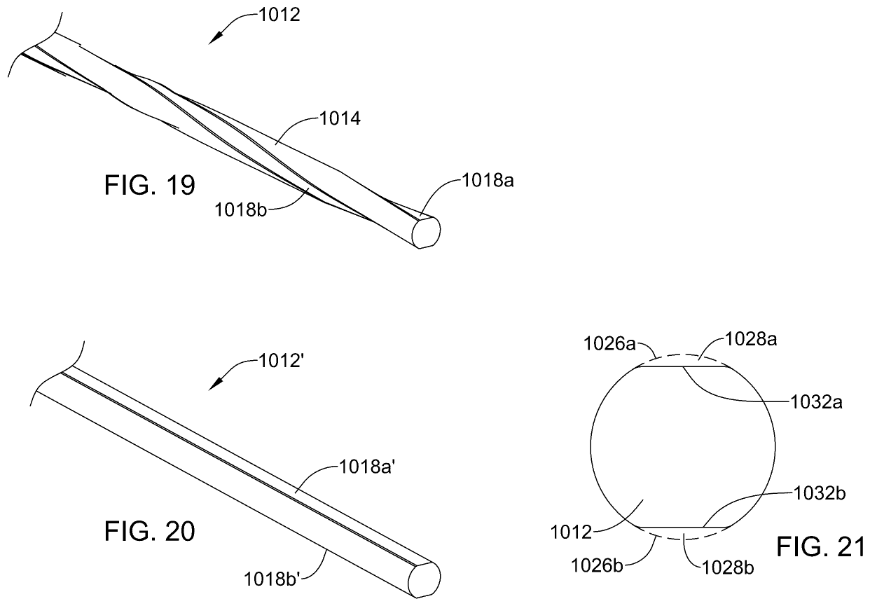

FIGS. 19-21 schematically illustrate a portion of a core wire 1012 that may be used, for example, in a suitable medical device such as those disclosed herein. In at least some instances, FIGS. 19-21 may illustrate a proximal end region 1014 of the core wire 1012. However, this is not intended to be limiting as the portion of the core wire 1012 may be essentially any suitable length or segment of the core wire 1012. As can be seen in FIG. 19 the proximal end region 1014 of the core wire 1012 may include a plurality of circular segment cutouts including circular segment cutouts 1018a, 1018b. The circular segment cutouts 1018a, 1018b may extend along a length of the core wire 1012. For example, the circular segment cutouts 1018a, 1018b may extend helically about the core wire 1012. Alternatively, the circular segment cutouts 1018a′, 1018b′ may extend axially along the core wire 1012′ as shown in FIG. 20.

In at least some instances, the proximal end region 1014 of the core wire 1012 may be formed from a shaft or wire that has a substantially circular cross-sectional shape. For example, in FIG. 21, reference numbers 1026a, 1026b corresponds to the shape or perimeter of the core wire 1012 prior to forming/cutting the circular segment cutouts 1018a, 1018b into the core wire 1012. The circular segment cutouts 1018a, 1018b may have a flattened outward-facing surface, denoted by the chord 1032a, 1032b. The portions of the core wire 1012 that are removed to form/cut the circular segment cutouts 1018a, 1018b in the core wire 1012 is labeled with reference number 1028a, 1028b. In other instances, the shape of the proximal end region 1014 may be achieved by molding, extruding, casting, mechanically working, or otherwise forming the proximal end region 1014 into the desired shape. In some instances, the proximal end region 1014 may include an outer coating with a tacky or textured consistency or feel.

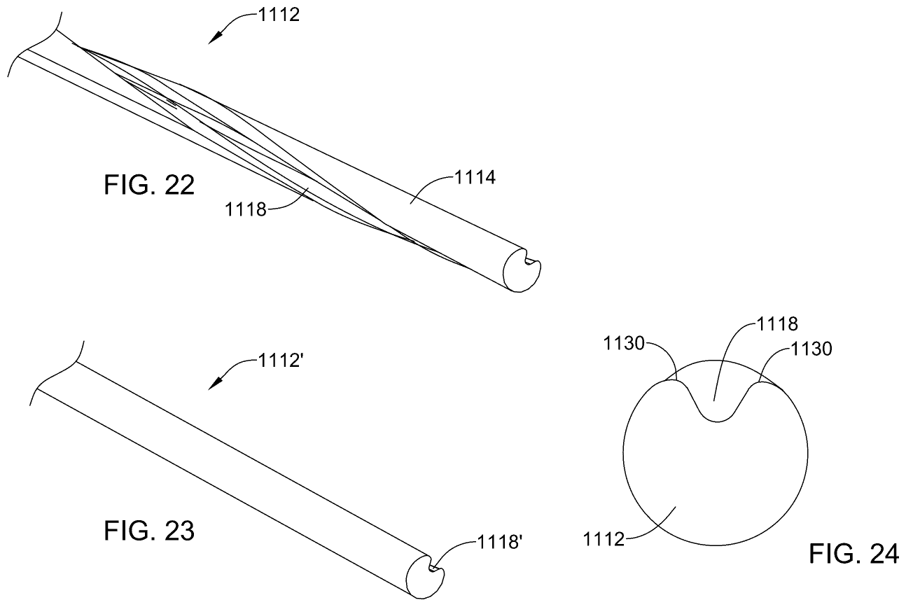

FIGS. 22-24 schematically illustrate a portion of a core wire 1112 that may be used, for example, in a suitable medical device such as those disclosed herein. In at least some instances, FIGS. 22-24 may illustrate a proximal end region 1114 of the core wire 1112. However, this is not intended to be limiting as the portion of the core wire 1112 may be essentially any suitable length or segment of the core wire 1112. As can be seen in FIG. 22, the proximal end region 1114 of the core wire 1112 may include a groove 1118. The groove 1118 may extend along a length of the core wire 1112. For example, the groove 1118 may extend helically about the core wire 1112. Alternatively, the groove 1118′ may extend axially along the core wire 1112′ as shown in FIG. 23.

In at least some instances, the proximal end region 1114 of the core wire 1112 may be formed from a shaft or wire that has a substantially circular cross-sectional shape. The groove 1118 may be formed by cutting out a portion of the core wire 1112 as can be seen in FIG. 24. The cutout forming the groove 1118 may have rounded edges or corners 1130. In other instances, the shape of the proximal end region 1114 may be achieved by molding, extruding, casting, mechanically working, or otherwise forming the proximal end region 1114 into the desired shape. In some instances, the proximal end region 1114 may include an outer coating with a tacky or textured consistency or feel.

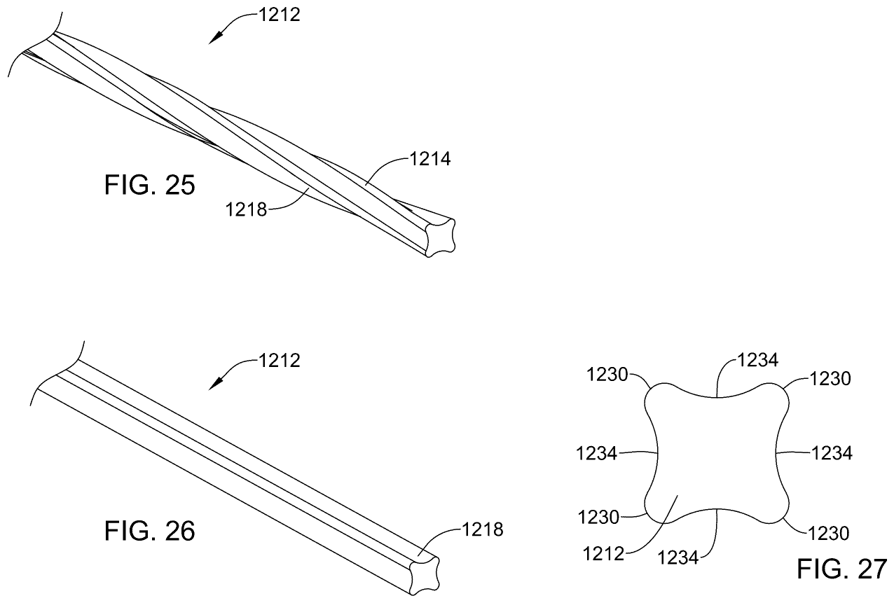

FIGS. 25-27 schematically illustrate a portion of a core wire 1212 that may be used, for example, in a suitable medical device such as those disclosed herein. In at least some instances, FIGS. 25-27 may illustrate a proximal end region 1214 of the core wire 1212. However, this is not intended to be limiting as the portion of the core wire 1212 may be essentially any suitable length or segment of the core wire 1212. As can be seen in FIG. 25, the proximal end region 1214 of the core wire 1212 may have a geometric shape. In this example, the proximal end region 1214 of the core wire 1212 has a squared shape with rounded corners 1230 and recessed sides 1234 as depicted in FIG. 27. In some instances, the proximal end region 1214 may have a twisted or helical configuration as shown in FIG. 25. Alternatively, the proximal end region 1214 may have a straight configuration as shown in FIG. 26.

In at least some instances, the proximal end region 1214 of the core wire 1212 may be formed from a shaft or wire that has a substantially circular cross-sectional shape that is cut to form the proximal end region 1214 into the desired shape. Material may be removed from the core wire 1212 using a suitable process such as laser cutting, mechanically (e.g., saw) cutting, etching, etc. After removing the material, the proximal end region 1214 may have the desired shape. This may provide a desirable level of feel, grip, tactile response, and/or the like. In other instances, the shape of the proximal end region 1214 may be achieved by molding, extruding, casting, mechanically working, or otherwise forming the proximal end region 1214 into the desired shape. In some instances, the proximal end region 1214 may include an outer coating with a tacky or textured consistency or feel.

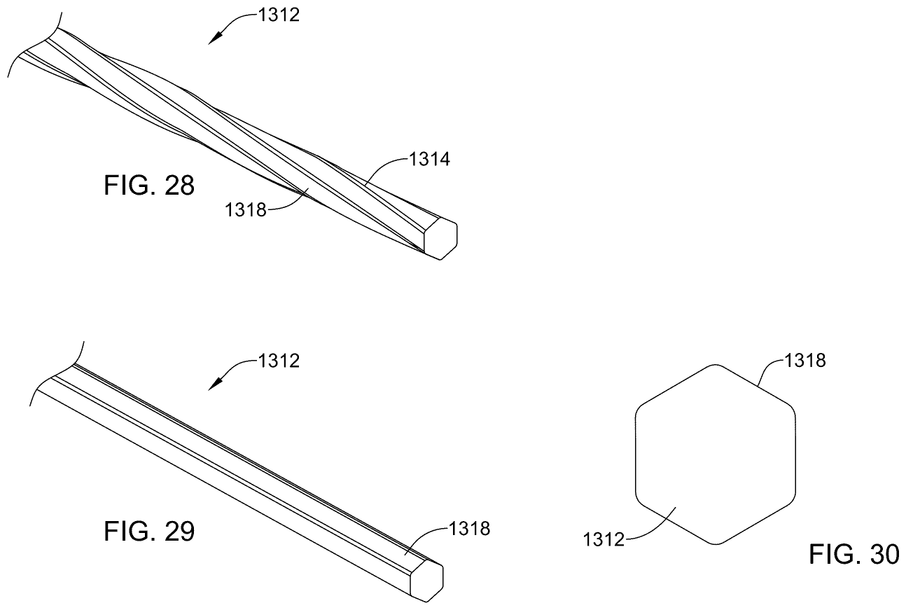

FIGS. 28-30 schematically illustrate a portion of a core wire 1312 that may be used, for example, in a suitable medical device such as those disclosed herein. In at least some instances, FIGS. 28-30 may illustrate a proximal end region 1314 of the core wire 1312. However, this is not intended to be limiting as the portion of the core wire 1312 may be essentially any suitable length or segment of the core wire 1312. As can be seen in FIG. 28, the proximal end region 1314 of the core wire 1312 may have a plurality of cutouts 1318 that form a geometric shape. In this example, the proximal end region 1314 of the core wire 1312 has a hexagonal shape. In some instances, the proximal end region 1314 may have a twisted or helical configuration as shown in FIG. 28. Alternatively, the proximal end region 1314 may have a straight configuration as shown in FIG. 29.

In at least some instances, the proximal end region 1314 of the core wire 1312 may be formed from a shaft or wire that has a substantially circular cross-sectional shape that is cut to form the proximal end region 1314 into the desired shape. Material may be removed from the core wire 1312 using a suitable process such as laser cutting, mechanically (e.g., saw) cutting, etching, etc. After removing the material, the proximal end region 1314 may have the desired shape. This may provide a desirable level of feel, grip, tactile response, and/or the like. In other instances, the shape of the proximal end region 1314 may be achieved by molding, extruding, casting, mechanically working, or otherwise forming the proximal end region 1314 into the desired shape. In some instances, the proximal end region 1314 may include an outer coating with a tacky or textured consistency or feel.

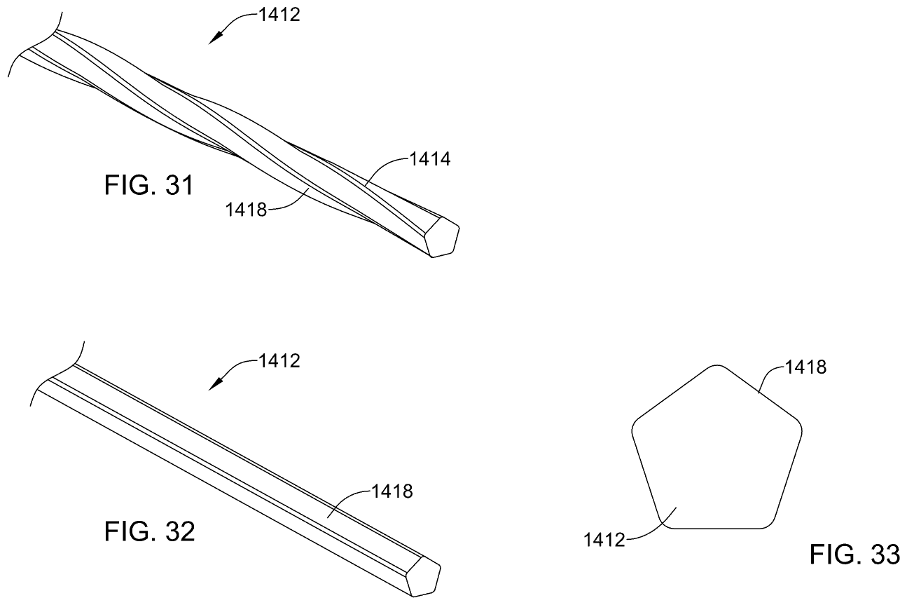

FIGS. 31-33 schematically illustrate a portion of a core wire 1412 that may be used, for example, in a suitable medical device such as those disclosed herein. In at least some instances, FIGS. 31-33 may illustrate a proximal end region 1414 of the core wire 1412. However, this is not intended to be limiting as the portion of the core wire 1412 may be essentially any suitable length or segment of the core wire 1412. As can be seen in FIG. 31, the proximal end region 1414 of the core wire 1412 may have a plurality of cutouts 1418 that form a geometric shape. In this example, the proximal end region 1414 of the core wire 1312 has a pentagonal shape. In some instances, the proximal end region 1414 may have a twisted or helical configuration as shown in FIG. 31. Alternatively, the proximal end region 1414 may have a straight configuration as shown in FIG. 32.

In at least some instances, the proximal end region 1414 of the core wire 1412 may be formed from a shaft or wire that has a substantially circular cross-sectional shape that is cut to form the proximal end region 1414 into the desired shape. Material may be removed from the core wire 1412 using a suitable process such as laser cutting, mechanically (e.g., saw) cutting, etching, etc. After removing the material, the proximal end region 1314 may have the desired shape. This may provide a desirable level of feel, grip, tactile response, and/or the like. In other instances, the shape of the proximal end region 1414 may be achieved by molding, extruding, casting, mechanically working, or otherwise forming the proximal end region 1414 into the desired shape. In some instances, the proximal end region 1414 may include an outer coating with a tacky or textured consistency or feel.

The materials that can be used for the various components of the medical device 10, 110 (and/or the components thereof) may include those commonly associated with medical devices. For simplicity purposes, the following discussion makes reference to the core wire 12 and other components of the medical device 10, 110. However, this is not intended to limit the devices and methods described herein, as the discussion may be applied to other similar tubular members and/or components of tubular members or devices disclosed herein.

The core wire 12 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10, 110 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10, 110 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10, 110 to achieve the same result.

In some embodiments, a degree of Magnetic Resonance Imaging (MRI) compatibility is imparted into the medical device 10, 110. For example, the medical device 10, 110, or portions thereof, may be made of a material that does not substantially distort the image and create substantial artifacts (e.g., gaps in the image). Certain ferromagnetic materials, for example, may not be suitable because they may create artifacts in an MRI image. The medical device 10, 110, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device with tactile feel, comprising:
an elongate shaft having a proximal end region and a distal end region;
wherein the proximal end region of the elongate shaft has a substantially circular cross-sectional shape; and
wherein the proximal end region includes a circular segment cutout formed therein, the circular segment cutout having a length extending along at least a portion of the proximal end region.

2. The medical device of claim 1, wherein the proximal end region and the distal end region are formed from a single monolith of material.

3. The medical device of claim 1, wherein the proximal end region includes a nickel-titanium alloy.

4. The medical device of claim 1, wherein the proximal end region includes stainless steel.

5. The medical device of claim 1, wherein a polymer tip member is disposed along the distal end region.

6. The medical device of claim 1, wherein a spring tip member is disposed along the distal end region.

7. The medical device of claim 1, wherein the circular segment cutout extends helically about the proximal end region.

8. The medical device of claim 1, wherein the circular segment cutout extends axially along the proximal end region.

9. The medical device of claim 1, wherein the proximal end region includes an outer coating.

10. A guidewire, comprising:
an elongate core wire having a proximal end region and a distal end region;
wherein the proximal end region includes a circular segment cutout formed therein, the circular segment cutout extending along at least a length of the proximal end region; and
a tip member coupled to the distal end region.

11. The guidewire of claim 10, wherein the elongate core wire is formed from a single monolith of material.

12. The guidewire of claim 10, wherein the elongate core wire includes a nickel-titanium alloy.

13. The guidewire of claim 10, wherein the elongate core wire includes stainless steel.

14. The guidewire of claim 10, wherein the tip member includes a polymer tip member disposed along the distal end region.

15. The guidewire of claim 10, wherein the tip member includes a spring tip member disposed along the distal end region.

16. The guidewire of claim 10, wherein the circular segment cutout extends helically about the proximal end region.

17. The guidewire of claim 10, wherein the circular segment cutout extends axially along the proximal end region.

18. A method for manufacturing a guidewire, the method comprising:
forming a circular segment cutout in a proximal end region of an elongate core wire;
wherein forming the circular segment cutout includes extending the circular segment cutout along at least a length of the proximal end region of the elongate core wire; and
coupling a tip member to a distal end region of the elongate core wire.

19. The method of claim 18, wherein forming the circular segment cutout includes extending the circular segment cutout helically about the proximal end region.

20. The method of claim 18, wherein forming the circular segment cutout includes extending the circular segment cutout axially along the proximal end region.
